(19) United States
(12) Patent Application Publication
(10) Pub. No.: US 2024/0138705 A1
(43) Pub. Date: May 2, 2024

(54) SYSTEMS AND METHODS FOR DETECTING APNEAS AND HYPONEAS

(71) Applicant: The Alfred E. Mann Foundation for Scientific Research, Valencia, CA (US)

(72) Inventors: Brian M. SHELTON, Altadena, CA (US); Sahar Elyahoodayan, Los Angeles, CA (US); Hemang Trivedi, San Jose, CA (US)

(21) Appl. No.: 18/495,627

(22) Filed: Oct. 26, 2023

Related U.S. Application Data
(60) Provisional application No. 63/381,046, filed on Oct. 26, 2022.

(51) Int. Cl. A61B 5/08; A61B 5/00; A61B 5/113

(52) U.S. Cl. CPC A61B 5/0826; A61B 5/0022; A61B 5/0031; A61B 5/0803; A61B 5/0816; A61B 5/113; A61B 5/4815; A61B 5/4836; A61B 5/686; A61B 5/721; A61B 5/7253; A61B 5/7267; A61B 5/7282; A61B 5/7292; A61B 5/742; A61B 5/0205; A61B 2562/0204; A61B 2562/0219; A61B 2562/0247

(57) ABSTRACT

The present disclosure generally relates to systems and methods for detecting and/or monitoring respiratory events (e.g., apnea and hypopneas) experienced by a subject during sleep and/or for generating a sleep quality metric for an individual, using one or more implanted or external sensors, as well as methods of treating medical conditions related thereto, such as obstructive sleep apnea.

### CROSS-REFERENCE TO RELATED APPLICATION

The application claims the benefit of U.S. Provisional Application No. 63/381,046, filed Oct. 26, 2022, which is herein incorporated by reference.

### TECHNICAL FIELD

The present disclosure generally relates to systems and methods for detecting apnea and/or hypopnea using one or more sensors, and methods of treating medical conditions related thereto.

### BACKGROUND

Obstructive Sleep Apnea (“OSA”) is a sleep disorder involving obstruction of the upper airway during sleep. The obstruction of the upper airway may be caused by the collapse of or increase in the resistance of the pharyngeal airway, often resulting from tongue obstruction. The obstruction of the upper airway may be caused by reduced genioglossus muscle activity during the deeper states of NREM sleep. Obstruction of the upper airway may cause breathing to pause during sleep. Cessation of breathing may cause a decrease in the blood oxygen saturation level, which may eventually be corrected when the person wakes up and resumes breathing. The long-term effects of OSA include high blood pressure, heart failure, strokes, diabetes, headaches, and general daytime sleepiness and memory loss, among other symptoms.

OSA is extremely common and may have a prevalence similar to diabetes or asthma. Over 100 million people worldwide suffer from OSA, with about 25% of those people being treated. Continuous Positive Airway Pressure (“CPAP”) is a conventional therapy for people who suffer from OSA. More than five million patients own a CPAP machine in North America, but many do not comply with use of these machines because they cover the mouth and nose and, hence, are cumbersome and uncomfortable.

Neurostimulators may be used to open the upper airway as a treatment for alleviating apneic events. Such therapy may involve stimulating the nerve fascicles of the hypoglossal nerve (“HGN”) that innervate the intrinsic and extrinsic muscles of the tongue in a manner that prevents retraction of the tongue which would otherwise close the upper airway during the inspiration period of the respiratory cycle. For example, current stimulator systems may be used to stimulate the trunk of the HGN with a nerve cuff electrode. However, these systems do not provide a sensor or sensing capabilities, and therefore, the stimulation delivered to the HGN trunk is not synchronized to the respiratory cycle or modulated based upon respiratory events experienced by the subject being treated.

### BRIEF SUMMARY

Ideally, a system for treating OSA should be able to account for respiratory events experienced by the individual being treated, and/or a metric indicative of the number or severity of respiratory events, such as the Apnea-Hypopnea Index (“AHI”) of the subject being treated. An OSA stimulation system configured to detect apnea or hypopnea, and/or to compute metrics related thereto, could inform the subject (and medical professionals) about the performance of a given therapy (e.g., on a nightly, weekly, or other basis). Moreover, such systems could tailor treatment based upon the detection of respiratory events or related metrics by auto-titrating settings such as stimulation intensity (e.g., by adjusting the pulse amplitude and/or pulse width of stimulation). In doing so, such systems would provide better patient care and provide a tool for monitoring the effectiveness of different therapy regiments or parameters, increasing the likelihood of an improved therapeutic outcome for the subject being treated.

The present disclosure addresses these and other shortcomings by providing OSA stimulation systems that can accurately detect and/or monitor respiratory events experienced by a subject being treated (e.g., to generate an AHI for the subject) using one or more implanted or external sensors incorporated into or in communication with the system. Such systems may advantageously be used to provide tailored treatment for a subject, to evaluate different stimulation regimens or parameters, and/or additional functionality compared to current systems, among other benefits which will become apparent in view of the following description and the accompanying figures. For example, the present disclosure provides systems and methods wherein stimulation intensity is auto-titrated upwards during a respiratory event, helping to mitigate the effects of respiratory events experienced by individuals suffering from OSA.

In a first general aspect, the disclosure provides a computer-implemented system for treating OSA in a human subject, comprising: one or more sensors, wherein each sensor is configured to collect sensor data indicative of respiratory activity and/or a physical state of the human subject when placed on, in proximity to, or implanted within, the human subject, and wherein the one or more sensors includes at least one implanted sensor; and a controller comprising a processor and memory, communicatively linked to the one or more sensors and configured to receive the sensor data from the one or more sensors, detect a respiratory event experienced by the subject, using the sensor data, and classify the detected respiratory event, using a trained classifier comprising an electronic representation of a classification system; and a stimulation system, communicatively linked to the controller and configured to deliver stimulation to a nerve which innervates an upper airway muscle of the human subject based on the classification by the controller.

In some aspects, the controller is configured to classify the detected respiratory event as normal breathing, an apnea event, or a hypopnea event. In some aspects, the one or more sensors each comprise: a pressure sensor, an accelerometer, an acoustic sensor, a gyroscope, an auscultation sensor, a heart rate monitor, an electrocardiogram (“ECG”) sensor, a blood pressure sensor, a blood oxygen level sensor, an electromyography (“EMG”) sensor, and/or a muscle sympathetic nerve activity (“MSNA”) sensor

In some aspects, the controller is further configured to generate a sleep quality metric for the human subject, wherein the sleep quality metric is based on the number of detected apnea or hypopnea events experienced by the subject. The sleep quality metric may be, e.g., an Apnea-Hypopnea Index (“AHI”), a Respiratory Disturbance Index (“RDI”), or a Respiratory Event Index (REI).

In some aspects, the one or more sensors comprises a sub-clavically implanted inertial measurement unit (“IU”).

In some aspects, the controller is located within a housing implanted in the human subject, and configured to predict an airflow reduction amount and an oxygen desaturation level for the human subject using sensor data obtained from the one or more sensors.

In some aspects, the one or more sensors comprises an acoustic sensor configured to detect a respiratory activity signal when positioned on, within, or in proximity to the chest, bronchi, or trachea of the human subject, and wherein the controller is further configured to apply a filter to the respiratory activity signal, wherein the filter is configured to reduce or eliminate a component of the respiratory activity signal caused by the human subject's heartbeat and/or snoring activity. In some aspects, the filter comprises a Hilbert transform and the controller is configured to apply an adaptive threshold, using the trained classifier, to identify regions of the respiratory signal corresponding to apnea and/or hypopnea events.

In some aspects, the one or more sensors comprises an IMU configured to detect motion by the human subject, and the controller is configured to identify one or more regions of the respiratory activity signal as a motion artifact based on detected motion by the human subject. Motion artifacts may, e.g., be ignored when computing an AHI or other sleep quality metric.

In some aspects, the trained classifier was trained using a baseline dataset, wherein the baseline dataset comprises: a) data generated during a prior single or multi-night polysomnography (“PSG”) study of the human subject; and/or b) data generated from a prior single or multi-night PSG study of a population of human subjects.

In some aspects, the system is configured to a) output the sleep quality metric to a graphical or text-based interface of an electronic device; and/or b) transmit the sleep quality metric to a local, remote or cloud-based server, or other electronic device.

In some aspects, the controller is configured to detect the respiratory event experienced by the subject using sensor data received from at least or exactly 1, 2, 3, 4, 5, 6, 7, 8, 9, or 10 sensors.

In some aspects, any of the functionality described herein may be performed by one controller (e.g., an implanted controller integrated into an implanted OSA stimulation system; or an external controller in a dedicated electronic device configured to wirelessly communicate with an implanted OSA stimulation system). It should be understood that the execution or performance of any of the individual functions or combinations of functions described herein may be split among multiple controllers. For example, computationally intensive functions such as classification may be offloaded to an external controller in order to conserve the battery life of an implanted OSA stimulation system. Thus, in some aspects, an implanted controller is configured to transmit sensor data to a local external controller or a remote external controller. A controller may execute an application, e.g., a user application or a clinician application, which acts as an interface to allow the user to view, modify, save, transmit, or otherwise interact with the OSA stimulation system, collected and/or processed sensor data, or any other parameters or features of the systems described herein.

In some aspects, the controller is configured to cause the stimulation system to apply, increase, decrease, temporarily pause, or terminate the stimulation based on the classification by the controller. For example, in some aspects the controller is configured to cause the stimulation system to change an amplitude, pulse width, and/or frequency of the stimulation based on the classification by the controller.

In a second general aspect, the disclosure provides methods for treating OSA using any of the systems described herein. For example, in some aspects such methods may comprise collecting sensor data indicative of respiratory activity and/or a physical state of the human subject, using one or more sensors configured to collect data when placed on, in proximity to, or implanted within, the human subject, wherein the one or more sensors includes at least one implanted sensor; receiving, by a controller comprising a processor and memory, the sensor data from the one or more sensors; detecting a respiratory event experienced by the subject, using the received sensor data; classifying the detected respiratory event, by the controller; wherein the controller is configured to perform the classification using a trained classifier comprising an electronic representation of a classification system, and/or to transmit the received sensor data to a server configured to perform the classification using a trained classifier comprising an electronic representation of a classification system.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

FIG. 1 is a diagram illustrating an exemplary embodiment of a system for treating OSA using a classifier configured to detect respiratory events (e.g., apnea or hypopnea events) based upon sensor data. In this example, the system comprises an external sensor, as well as an implanted sensor integrated into the housing of an implanted OSA stimulation system. Optional cloud-based components of a system according to the disclosure are also illustrated.

FIG. 2 is a conceptual flow diagram summarizing a method for detecting respiratory events (e.g., apnea or hypopnea events) using the systems described herein, and optionally the use of such systems to treat the subject for OSA.

FIG. 3 is a conceptual flow diagram summarizing another general method for detecting and classifying respiratory events (e.g., as apnea or hypopnea events) using the systems described herein, and optionally adjusting OSA stimulation based on the same.

FIG. 4 is an exemplary respiratory waveform illustrating a period of abnormal respiratory activity followed by a period of normal respiratory activity.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several aspects of exemplary embodiments according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” that includes one or more processors. Examples of processors include microprocessors, microcontrollers, graphics processing units (“GPUs”), central processing units (“CPUs”), application processors, digital signal processors (“DSPs”), reduced instruction set computing (“RISC”) processors, systems on a chip (“SoC”), baseband processors, field programmable gate arrays (“FPGAs”), programmable logic devices (“PLDs”), application-specific integrated circuits (“ASICs”), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (“RAM”), a read-only memory (ROM), an electrically erasable programmable ROM (“EEPROM”), optical disk storage, magnetic disk storage, other magnetic storage devices, combinations of the aforementioned types of computer-readable media, or any other medium that can be used to store computer executable code in the form of instructions or data structures that can be accessed by a computer.

As noted above, the present disclosure is generally directed to systems and methods for detecting and classifying respiratory events (e.g., apnea or hypopnea) and/or for computing related metrics, as well as methods of treating medical conditions related thereto. Sleep, and more specifically quality of sleep, is now recognized as critical to human health. Lack of sleep, or poor sleep contributes significantly not only to poor cognitive performance, but also to a host of human health conditions including hypertension, heart failure, cognitive disorders, diabetes, and many others. Accordingly, there exists a need in the art for the systems described herein, which provide new tools for monitoring and treating OSA, and which offer improved accuracy and convenience compared to current systems.

As used herein, the terms “apnea” and “hypopnea” are to be understood with reference to the current American Academy of Sleep Medicine (“AASM”) definitions for these terms. The AASM guidelines define a “hypopnea” (in an adult patient) as a ≥30% reduction in nasal pressure, nasal airflow, or other some other hypopnea sensor that last for ≥10 seconds and that corresponds to an oxygen desaturation event. A hypopnea oxygen desaturation event can either be defined as a ≥3% drop from the pre-event baseline with an arousal or as a ≥4% drop from the pre-event baseline without consideration of an arousal. The AASM guidelines further define an “apnea” as a cessation of airflow for >10 seconds.

As used herein, the term “Apnea-Hypopnea Index” (“AHI”) refers to a metric calculated by dividing the total number of apneas and hypopneas by the subject's total sleep time. It is understood that the AHI is an exemplary metric that may be used by the systems described herein. In some aspects, alternative metrics such as the “Respiratory Disturbance Index” (“RDI”) or “Respiratory Event Index” (“REI”) may be used. The RDI is defined as the total number of apneas, hypopneas and Respiratory Effort-Related Arousals (“RERAs”), divided by the subject's total sleep time in hours. The REI is defined as the total number of apneas and hypopneas, divided by the total monitoring time in hours. An AHI or REI<5 per hour is normal (for adults); an AHI or REI of 5-14.9 per hour is indicative of mild OSA; an AHI or REI of 15-29.9 per hour is indicative of moderate OSA; and an AHI or REI of >30 per hour is indicative of severe OSA.

Current systems for the detection of apnea and hypopnea suffer from various drawbacks. For example, the detection of hypopneas and apneas typically requires a combination of a nasal flow (or nasal temperature) sensor, a respiration effort (e.g., a thoracic RIP band) sensor, and an oximeter. Wearing a nasal flow sensor, a nasal temperature sensor, and a thoracic RIP band can be uncomfortable and requires user intervention. This is not only a patient burden, but can also disturb sleep. Moreover, such systems often depend upon a wired communication system (adding to patient discomfort) or a wireless system that may not fail to capture data from the various sensors or which may rely upon additional electronic devices that may not be present, or which could have become disconnected from the system.

In contrast, the OSA stimulation system described herein simplify and improve upon existing designs by incorporating additional sensor functionality into one or more implanted components of the system. Such systems may utilize, e.g., a combination of electrocardiogram (“ECG”) detection, optical SpO₂ detection, respiratory detection (e.g., using an implanted IMU), a microphone, and/or a heart rate sensor (e.g., to detect Heart Rate Variability, “HRV”), in order to detect apnea and hypopnea events and to compute a sleep quality metric (e.g., an AHI or an AHI-like metric). The sleep quality metric may in turn be used to auto-titrate stimulation intensity, (e.g., stimulation current, pulse width, or duty cycle). The sleep quality metric may also be used to find an optimum stimulation frequency or pulse width.

A system according to the disclosure may utilize one or more implanted sensors (e.g., incorporated into an implanted OSA stimulation system), without the need for sensor data collected from any external sensors. However, in some aspects, an implanted device may not have the sensors required to measure all of the parameters described herein. In such cases, one or more external sensors may be used to supplement the dataset available to the system, e.g., to include actigraphy, heart rate, blood oxygen level, blood pressure, EEG, single or low channel EEG, in-ear EEG, other brain activity like FNIRS (functional near infra-red spectroscopy), EMG, eye movement (EOG), and environmental data such a temperature, humidity, extraneous noise, etc. In that sense, it is understood that systems according to the disclosure are modular in that they may take into account sensor data collected using any combination of implanted and external sensors, and may further be configured to take into account parameters based on environmental data (e.g., temperature) and biomarker concentrations or amounts (e.g., determined based upon an analysis of a subject's blood).

In some aspects, the present disclosure contemplates using data from an active implantable device with or without supplemental data from one or more external devices to determine an AHI or AHI-like metric, which is a measure of sleep quality for a person on a given night. In some aspects, artificial intelligence, machine learning, and/or deep learning may be used to compare data collected from a patient with generalized datasets to develop or inform one or more classification algorithms (e.g., to allow for the detection and/or classification of respiratory events such as apneas and hypopneas). In another aspect, the data collected from a patient may be compared with data collected during a PSG study for a single (or multiple) nights to inform/train the respiratory event classification algorithm for that patient. In another aspect, data collected from an implanted sensor and one or more external sensors, obtained from several patients, can be compared with PSG data from these patients to more broadly inform a population-based respiratory event classification algorithm.

In this manner, once a classifier algorithm has been trained, it can subsequently be used to monitor for and detect respiratory events (e.g., apneas and hypopneas), allowing for the generation of an AHI or AHI-like sleep quality metric, and optionally, the modulation of stimulation parameters for a person being treated using the systems described herein. The computation used to generate a classification, which is typically based on the data collected for that night (but which may alternately include multiple days and nights), could be performed by an implanted component of an OSA stimulation system (e.g., a controller within the housing of an implant), by an external controller that is able to communicate with the OSA stimulation system, by an application that is able to communicate with the OSA stimulation system, or remotely (e.g., by a cloud-based or remote server).

In some aspects, the systems described herein may be configured to generate a numeric sleep quality metric as the primary output calculated. In some aspects, the sleep quality metric may be used to motivate the patient to be more compliant with their therapy by illustrating how their sleep (and/or other physiological parameters) improves when they use the device. It may also illustrate how certain behaviors (e.g., activity, alcohol consumption, overeating, salt consumption, late night snacking, afternoon napping, etc.) can impact sleep quality to motivate better behavior or lifestyle choices. In some aspects, a patient's sleep quality metric, or any individually-measured parameter(s) may be compared with other patients for whom data has been collected, in order to provide a rank or otherwise illustrate how they are similar or different to other people. This could be a comparison to all other patients, or patients that share one or more similar traits to the target patient (e.g., age, general health, blood pressure, degree of SDB, etc.). Awards, either virtual or tangible, could be given for improvements in behavior and/or scores.

In some aspects, the sleep quality metric may be provided to a user via an interface of the system or using an electronic device communicatively-linked to the system (e.g., a mobile application executed on a smart phone, tablet, or external controller wirelessly paired with the system). In some aspects, the system may be configured to also provide recommendations to the patient using an interface or paired electronic device use (e.g., to remind the user to turn on the OSA stimulation system, or to change the system's settings), and to suggest behavior modification(s) (e.g., advising a patient not to eat after a particular time, or to adjust the temperature of the room where they intend to sleep). The systems described herein may also be configured to alert a physician when intervention may be needed (e.g., a device fault is detected, or reprogramming may be required) and to offer to set up an appointment (e.g., in person or via telemedicine) for the patient. In some aspects, a physician is able to interrogate and program the system, and any of the implanted or external sensors remotely. In some aspects, a software application (or controller or other device configured to control the systems described herein) may also engage the patient in other ways. For example, it may collect information provided by the patient (e.g., questionnaires, polls), and/or it could offer to facilitate a post to social media about the patient's status that day, or satisfaction with the device. It may also collect voice data, including testimonials or observations.

As explained herein, the present systems may also be used in conjunction with a digital platform (e.g., local application(s) combined with edge and/or cloud-based processing and storage) to collect data from an implanted sensor (and optionally wearable devices), send the collected data to the cloud, compute sleep quality metrics locally or in the cloud, and share these insights with both the patient and a remote clinician. The sleep quality metric can be used to motivate the patient to take certain actions, including potentially adjusting settings on their implant, actively choosing to use their implant more, schedule visits with their medical provider, participate in a poll, and post to social media sites. Patient motivation may be in the form of a competition, comparing their scores to other patients, or other patients that are similar to them in some way, providing a ranking and showing how their score and/or their ranking have improved over time because of actions they have taken.

Classifiers

The term “classifier,” as used herein, refers broadly to a machine learning algorithm such as support vector machine(s), AdaBoost classifier(s), penalized logistic regression, elastic nets, regression tree system(s), gradient tree boosting system(s), naive Bayes classifier(s), neural nets, Bayesian neural nets, k-nearest neighbor classifier(s), deep learning systems, and random forest classifiers. The systems and methods described may use any of these classifiers, or combinations thereof.

A “Classification and Regression Tree” (“CART”), as used herein, refers broadly to a method to create decision trees based on recursively partitioning a data space so as to optimize one or more metrics, e.g., model performance.

The classification systems used herein may include computer executable software, firmware, hardware, or combinations thereof. For example, the classification systems may include reference to a processor and supporting data storage. Further, the classification systems may be implemented across multiple devices or other components local or remote to one another. The classification systems may be implemented in a centralized system, or as a distributed system for additional scalability. Moreover, any reference to software may include non-transitory computer readable media that when executed on a computer, causes the computer to perform one or more steps.

There are many potential classifiers that can be used by the systems and methods described herein. Machine and deep learning classifiers include but are not limited to AdaBoost, Artificial Neural Network (“ANN”) learning algorithms, Bayesian belief networks, Bayesian classifiers, Bayesian neural networks, Boosted trees, case-based reasoning, classification trees, Convolutional Neural Networks, decisions trees, Deep Learning, elastic nets, Fully Convolutional Networks (“FCN”), genetic algorithms, gradient boosting trees, k-nearest neighbor classifiers, LASSO, Linear Classifiers, naive Bayes classifiers, neural nets, penalized logistic regression, Random Forests, ridge regression, support vector machines, or an ensemble thereof. See, e.g., Han & Kamber (2006) Chapter 6, Data Mining, Concepts and Techniques, 2nd Ed. Elsevier: Amsterdam. As described herein, any classifier or combination of classifiers (e.g., an ensemble) may be used by the present systems.

Deep Learning Algorithms

In some aspects, the classifier is a deep learning algorithm. Machine learning is a subset of artificial intelligence that uses a machine's ability to take a set of data and learn about the information it is processing by changing the algorithm as data is being processed. Deep learning is a subset of machine learning that often utilizes artificial neural networks inspired by the workings on the human brain. For example, the deep learning architecture may be multilayer perceptron neural network (“MLPNN”), backpropagation, Convolutional Neural Network (“CNN”), Recurrent Neural Network (“RNN”), Long Short-Term Memory (“LSTM”), Generative Adversarial Network (“GAN”), Restricted Boltzmann Machine (“RBM”), Deep Belief Network (“DBN”), or an ensemble thereof.

Classification Trees

A classification tree is an easily interpretable classifier with built in feature selection. A classification tree recursively splits the data space in such a way so as to maximize the proportion of observations from one class in each subspace.

The process of recursively splitting the data space creates a binary tree with a condition that is tested at each vertex. A new observation is classified by following the branches of the tree until a leaf is reached. At each leaf, a probability is assigned to the observation that it belongs to a given class. The class with the highest probability is the one to which the new observation is classified. Classification trees are essentially a decision tree whose attributes are framed in the language of statistics. They are highly flexible but very noisy (the variance of the error is large compared to other methods).

Tools for implementing classification tree are available, by way of non-limiting example, for the statistical software computing language and environment, R. For example, the R package “tree,” version 1.0-28, includes tools for creating, processing and utilizing classification trees. Examples of Classification Trees include but are not limited to Random Forest. See also Kamiński et al. (2017) “A framework for sensitivity analysis of decision trees.” Central European Journal of Operations Research. 26(1): 135-159; Karimi & Hamilton (2011) “Generation and Interpretation of Temporal Decision Rules”, International Journal of Computer Information Systems and Industrial Management Applications, Volume 3, the content of which is incorporated by reference in its entirety.

Random Forest Classifiers

Classification trees are typically noisy. Random forests attempt to reduce this noise by taking the average of many trees. The result is a classifier whose error has reduced variance compared to a classification tree. Methods of building a Random Forest classifier, including software, are known in the art. Prinzie & Poel (2007) “Random Multiclass Classification: Generalizing Random Forests to Random MNL and Random NB.” Database and Expert Systems Applications. Lecture Notes in Computer Science. 4653; Denisko & Hoffman (2018) “Classification and interaction in random forests.” PNAS 115(8): 1690-1692, the contents of which are incorporated by reference in its entirety.

To classify a new observation using the random forest, classify the new observation using each classification tree in the random forest. The class to which the new observation is classified most often amongst the classification trees is the class to which the random forest classifies the new observation. Random forests reduce many of the problems found in classification trees but at the tradeoff of interpretability.

Tools for implementing random forests as discussed herein are available, by way of non-limiting example, for the statistical software computing language and environment, R. For example, the R package “random Forest,” version 4.6-2, includes tools for creating, processing and utilizing random forests.

AdaBoost (Adaptive Boostin)

AdaBoost provides a way to classify each of n subjects into two or more categories based on one k-dimensional vector (called a k-tuple) of measurements per subject. AdaBoost takes a series of “weak” classifiers that have poor, though better than random, predictive performance and combines them to create a superior classifier. The weak classifiers that AdaBoost uses are CARTs. CARTs recursively partition the dataspace into regions in which all new observations that lie within that region are assigned a certain category label. AdaBoost builds a series of CARTs based on weighted versions of the dataset whose weights depend on the performance of the classifier at the previous iteration. See Han & Kamber (2006) Data Mining, Concepts and Techniques, 2nd Ed. Elsevier: Amsterdam, the content of which is incorporated by reference in its entirety. AdaBoost technically works only when there are two categories to which the observation can belong. For g>2 categories, (g/2) models must be created that classify observations as belonging to a group of not. The results from these models can then be combined to predict the group membership of the particular observation. Predictive performance in this context is defined as the proportion of observations misclassified.

Convolutional Neural Network

Convolutional Neural Networks (“CNNs” or “ConvNets”) are a class of deep, feed-forward artificial neural networks, most commonly applied to analyzing visual imagery. CNNs use a variation of multilayer perceptrons designed to require minimal preprocessing. They are also known as shift invariant or space invariant artificial neural networks (“SIANN”), based on their shared-weights architecture and translation invariance characteristics. Convolutional networks were inspired by biological processes in that the connectivity pattern between neurons resembles the organization of the animal visual cortex. Individual cortical neurons respond to stimuli only in a restricted region of the visual field known as the receptive field. The receptive fields of different neurons partially overlap such that they cover the entire visual field. CNNs use relatively little pre-processing compared to other image classification algorithms This means that the network learns the filters that in traditional algorithms were hand-engineered. This independence from prior knowledge and human effort in feature design is a major advantage. LeCun and Bengio (1995) “Convolutional networks for images, speech, and time-series,” in Arbib (Ed.), The Handbook of Brain Theory and Neural Networks, MIT Press, the content of which is incorporated by reference in its entirety. Fully convolutional indicates that the neural network is composed of convolutional layers without any fully connected layers or MLP usually found at the end of the network. Convolutional Neural Network is an example of deep learning.

Support Vector Machines

Support vector machines (“SVMs”) are recognized in the art. In general, SVMs provide a model for use in classifying each of n subjects to two or more categories based on one k-dimensional vector (called a k-tuple) per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction. Vapnik (1998) Statistical Learning Theory; Vapnik “An overview of statistical learning theory” IEEE Transactions on Neural Networks 10(5): 988-999 (1999) the content of which is incorporated by reference in its entirety. Any new observation is then classified as belonging to any one of the categories of interest, based where the observation lies in relation to the hyperplane. When more than two categories are considered, the process is carried out pairwise for all of the categories and those results combined to create a rule to discriminate between all the categories. See Cristianini, N., & Shawe-Taylor, J. (2000). An Introduction to Support Vector Machines and Other Kernel-based Learning Methods. Cambridge: Cambridge University Press provides some notation for support vector machines, as well as an overview of the method by which they discriminate between observations from multiple groups.

In an exemplary embodiment, a kernel function known as the Gaussian Radial Basis Function (RBF) is used. Vapnik, 1998. The RBF may be used when no a priori knowledge is available with which to choose from a number of other defined kernel functions such as the polynomial or sigmoid kernels. See Han et al. Data Mining: Concepts and Techniques, Morgan Kaufman 3rd Ed. (2012). The RBF projects the original space into a new space of infinite dimension. A discussion of this subject and its implementation in the R statistical language can be found in Karatzoglou et al. “Support Vector Machines in R,” Journal of Statistical Software 15(9) (2006), the content of which is incorporated by reference in its entirety. SVM statistical computations may be performed using the statistical software programming language and environment R 2.10.0 (e.g., SVMs may be fitted using the ksvm( ) function in the kernlab package). Other suitable kernel functions include, but are not limited to, linear kernels, radial basis kernels, polynomial kernels, uniform kernels, triangle kernels, Epanechnikov kernels, quartic (biweight) kernels, tricube (triweight) kernels, and cosine kernels. Support vector machines are one out of many possible classifiers that could be used on the data. By way of non-limiting example, and as discussed below, other methods such as naive Bayes classifiers, classification trees, k-nearest neighbor classifiers, etc., may be used on the same data used to train and verify the support vector machine.

Naïve Bayes Classifier

The set of Bayes Classifiers are a set of classifiers based on Bayes' Theorem. See, e.g., Joyce (2003), Zalta, Edward N. (ed.), “Bayes' Theorem”, The Stanford Encyclopaedia of Philosophy (Spring 2019 Ed.), Metaphysics Research Lab, Stanford University, the content of which is incorporated by reference in its entirety.

Classifiers of this type seek to find the probability that an observation belongs to a class given the data for that observation. The class with the highest probability is the one to which each new observation is assigned. Theoretically, Bayes classifiers have the lowest error rates amongst the set of classifiers. In practice, this does not always occur due to violations of the assumptions made about the data when applying a Bayes classifier.

The naïve Bayes classifier is one example of a Bayes classifier. It simplifies the calculations of the probabilities used in classification by making the assumption that each class is independent of the other classes given the data. Naïve Bayes classifiers are used in many prominent anti-spam filters due to the ease of implantation and speed of classification but have the drawback that the assumptions required are rarely met in practice. Tools for implementing naive Bayes classifiers as discussed herein are available for the statistical software computing language and environment, R. For example, the R package “e1071,” version 1.5-25, includes tools for creating, processing and utilizing naive Bayes classifiers.

Neural Networks

One way to think of a neural network is as a weighted directed graph where the edges and their weights represent the influence each vertex has on the others to which it is connected. There are two parts to a neural network: the input layer (formed by the data) and the output layer (the values, in this case classes, to be predicted). Between the input layer and the output layer is a network of hidden vertices. There may be, depending on the way the neural network is designed, several vertices between the input layer and the output layer.

Neural networks are widely used in artificial intelligence and data mining but there is the danger that the models the neural nets produce will over fit the data (i.e., the model will fit the current data very well but will not fit future data well). Tools for implementing neural nets as discussed herein are available for the statistical software computing language and environment, R. For example, the R package “e1071,” version 1.5-25, includes tools for creating, processing and utilizing neural nets.

k-Nearest Neighbor Classifiers (KNN)

The nearest neighbor classifiers are a subset of memory-based classifiers. These are classifiers that have to “remember” what is in the training set in order to classify a new observation. Nearest neighbor classifiers do not require a model to be fit.

To create a k-nearest neighbor (knn) classifier, the following steps are taken:
1. Calculate the distance from the observation to be classified to each observation in the training set. The distance can be calculated using any valid metric, though Euclidian and Mahalanobis distances are often used. The Mahalanobis distance is a metric that takes into account the covariance between variables in the observations.
2. Count the number of observations amongst the k nearest observations that belong to each group.
3. The group that has the highest count is the group to which the new observation is assigned.

Nearest neighbor algorithms have problems dealing with categorical data due to the requirement that a distance be calculated between two points but that can be overcome by defining a distance arbitrarily between any two groups. This class of algorithm is also sensitive to changes in scale and metric. With these issues in mind, nearest neighbor algorithms can be very powerful, especially in large data sets. Tools for implementing k-nearest neighbor classifiers as discussed herein are available for the statistical software computing language and environment, R. For example, the R package “e1071,” version 1.5-25, includes tools for creating, processing and utilizing k-nearest neighbor classifiers.

Training Data

In another aspect, methods described herein include training of about 75%, about 80%, about 85%, about 90%, or about 95% of the data in the library or database and testing the remaining percentage for a total of 100% data. In an aspect, from about 70% to about 90% of the data is trained and the remainder of about 10% to about 30% of the data is tested, from about 80% to about 95% of the data is trained and the remainder of about 5% to about 20% of the data is tested, or from about 90% of the data is trained and the remainder of about 10% of the data is tested.

In some aspects, the database or library contains data from the analysis of over about 25, about 60, over about 125, over about 250, over about 500, or over about 1000 human subjects (collected using systems according to the disclosure, PSG studies, etc.). In some aspects, the data may comprise data from healthy subjects and/or from those known to have OSA.

The training data may comprise, e.g., data relating to any of the parameters described herein, including sensor data, biomarker data, environmental data, or any combinations thereof.

Methods of Classification

The disclosure provides for methods of classifying data (e.g., sensor data from one or more implanted or external sensors) obtained from an individual in order to generate a sleep quality metric (e.g., an AHI or AHI-like metric). In some aspects, these methods involve preparing or obtaining training data, as well as evaluating test data obtained from an individual (as compared to the training data), using one of the classification systems including at least one classifier as described above. Preferred classification systems use classifiers such as, but not limited to, support vector machines (“SVMs”), AdaBoost, penalized logistic regression, naive Bayes classifiers, classification trees, k-nearest neighbor classifiers, Deep Learning classifiers, neural nets, random forests, Fully Convolutional Networks (“FCN”), Convolutional Neural Networks (“CNN”), and/or an ensemble thereof. Deep Learning classifiers are a more preferred classification system. The classification system may be configured, e.g., to detect and/or measure respiratory events (e.g., apneas and hypopneas), based on sensor data collected from one or more implanted sensors, one or more external sensors, or combinations thereof.

As noted above, in some aspects a classifier may comprise an ensemble of multiple classifiers. For example, an ensemble method may include SVM, AdaBoost, penalized logistic regression, naive Bayes classifiers, classification trees, k-nearest neighbor classifiers, neural nets, Fully Convolutional Networks (FCN), Convolutional Neural Networks (CNN), Random Forests, deep learning, or any ensemble thereof, in order to make any of the determinations described herein.

An exemplary method for classifying sleep stage and/or quality may comprise the steps of: (a) accessing an electronically stored set of training data vectors, each training data vector or k-tuple representing an individual human subject and comprising sensor data for the respective human subject for each replicate, the training data vector further comprising a classification with respect to a respiratory event (e.g., an apnea or hypopnea event) experienced by the respective human subject; (b) training an electronic representation of a classifier or an ensemble of classifiers as described herein using the electronically stored set of training data vectors; (c) receiving test data comprising a plurality of sensor data for a test subject; (d) evaluating the test data using the electronic representation of the classifier and/or an ensemble of classifiers as described herein; and (e) outputting a classification as to one or more respiratory events experienced by the test subject, based on the evaluating step (e.g., detecting one or more apnea or hypopnea events). The test subject may be the same as the human subject used for training purposes (e.g., a baseline may be established for an individual using past data). In some aspects, the system will instead be trained with sensor data obtained from a plurality of human subjects (e.g., a population which may contain healthy individuals known not to have OSA, individuals known to have OSA, or a combination thereof).

In another embodiment, the disclosure provides a method of classifying test data, the test data comprising sensor data for a test subject, comprising: (a) accessing an electronically stored set of training data vectors, each training data vector or k-tuple representing an individual human subject and comprising sensor data for the respective human subject for each replicate, the training data further comprising a classification with respect to one or more respiratory events (e.g., apneas or hypopneas) experienced by the respective human subject; (b) using the electronically stored set of training data vectors to build a classifier and/or ensemble of classifiers; (c) receiving test data comprising a plurality of sensor data for a human test subject; (d) evaluating the test data using the classifier(s); and (e) outputting a classification as to one or more respiratory events (e.g., apnea or hypopnea events) experienced by the human test subject, based on the evaluating step. Alternatively, all (or any combination of) the replicates may be averaged to produce a single value. Outputting in accordance with this invention includes displaying information regarding the classification of the human test subject in an electronic display in human-readable form. The sensor data and/or biometric data may comprise data in accordance with any of the exemplary aspects of the present systems and methods described herein. In some aspects, the set of training vectors may comprise at least 20, 25, 30, 35, 50, 75, 100, 125, 150, or more vectors.

Classifier-Based Systems and Methods

As explained above, the systems and methods provided herein may be used to detect respiratory events experienced by a human subject while asleep, and to generate a sleep quality metric (e.g., an AHI or AHI-like metric), and optionally to treat OSA (e.g., by auto-titrating stimulation parameters when a respiratory event is detected). OSA stimulation systems according to the disclosure possess several advantages compared to prior systems, and in particular allow for more accurate tailoring of stimulation. Such systems also provide a convenient option for monitoring the effectiveness of a therapeutic regimen or parameters. Moreover, the present systems are advantageous in that they do not require invasive or uncomfortable sensors, improving the likelihood of patient compliance and positive therapeutic outcomes.

In some aspects, respiratory events (e.g., apneas and hypopneas) may be monitored by an implanted OSA stimulation system configured to communicate with an external device that performs this monitoring function (e.g., using one or more sensors), such as a smart watch or health tracker, if the patient already wears this device while asleep. However, in other aspects, is preferable for an implanted OSA stimulation system to detect and classify these events without communicating with any external device. One potential solution would be to incorporate an SpO₂ sensor into the implant, e.g., via using an optical sensor in an IR/Red transparent epoxy header.

All of the criteria for hypopnea and apnea detection are present in a respiratory effort signal. See Bianchi et al., “Automated Sleep Apnea Quantification Based on Respiratory Movement” (Int. J. Med. Sci. 2014; 11(8): 796-802), incorporated herein by reference. For example, a restriction in the airway would be associated with increased back-pressure and would impact the measurement of respiratory effort, e.g., the chest would not move as much if the airway is blocked. Likewise, in order to have a desaturation event, either the effort would have to be reduced (as in central sleep apnea) or the airway would have to be blocked. Both ECG and acoustic sensors have been used to detect respiratory effort and effect and consequently apnea and hypopnea events. See Mostafa et al., “A Systematic Review of Detecting Sleep Apnea Using Deep Learning.” Sensors 2019, 19, 4934.

In some aspects, the systems described herein use a sub-clavically implanted IMU to monitor respiration effort, allowing for the generation of an AHI-like indicator of respiration events. Ideally, the analysis of the respiration signal would be performed by an implanted OSA stimulation system (e.g., by a controller module of such a system) in order to predict airflow reduction amount, oxygen desaturation, hypopneas, and apneas. However, since this analysis may require considerable energy, a more energy-conservative approach would be to only identify effort, (i.e., respiration intensity corrected for patient orientation and respiration rate), in the implant. In that case, the raw data for the suspected respiration events could then be uploaded to the cloud for further analysis. As an alternative, all of the raw respiration data may be uploaded to the cloud for analysis (e.g., throughout the night or in one upload in the morning after the subject has completed a sleeping session). Then, analysis of the raw data may be performed in the cloud where there is no need for energy conservation.

In some aspects, a system according to the disclosure may utilize an acoustic sensor on, within, or in proximity to the chest, bronchi, or trachea to detect respiratory activity and classify it as normal breathing, apnea or hypopnea. To do so, the initial sound signal collected from the acoustic sensor may be filtered to remove artifacts such as heartbeat and snoring. An envelope extraction technique such as Hilbert transform may then be applied to find envelopes of the signal that correspond to frequencies below, e.g., 0.5 Hz (the typical frequency range of respiratory activity). An adaptive threshold may then be applied by a classifier, using a machine learning algorithm, to identify regions of the processed signal corresponding to sleep events versus normal breathing. The threshold may be continuously updated to classify changes in amplitude.

Artifacts such as drops in signal amplitude as a subject moves around may occur throughout night and can be mistakenly classified as an apneic event. This is because subjects may hold their breath or there may be a temporary loss in the signal during movement. As such, in some aspects an IMU-based motion sensor may be used to identify motion artifacts, e.g., so that regions of the processed signal that correspond to motion artifacts may be disregarded when calculating the number of apneas and hypopneas per hour, or other sleep quality metrics. Thus, in some aspects a system that incorporates a combination of sound and motion sensors may provide superior classification accuracy.

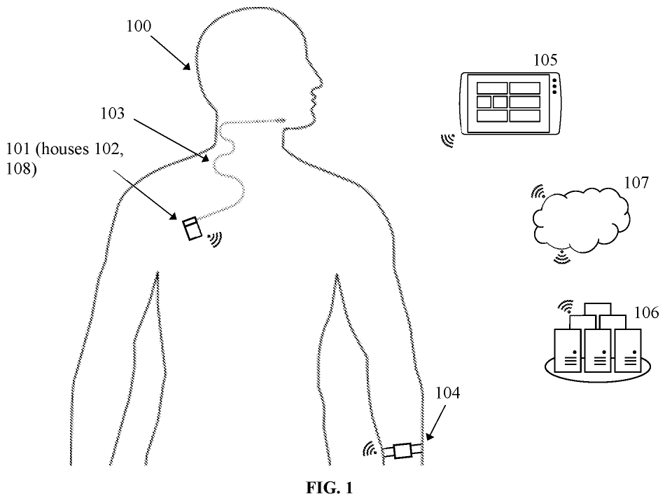

FIG. 1 is a diagram illustrating an exemplary embodiment of a system for treating OSA (100) using a classifier configured to detect respiratory events (e.g., apnea or hypopnea events) based upon sensor data. In this example, the system comprises an external sensor (104), as well as an implanted controller (102) and implanted sensor (108) integrated into the housing of an implanted OSA stimulation system (101). An external controller (105) and optional cloud-based components (106, 107) of a system according to the disclosure are also illustrated.

In this example, the system comprises two sensors (104, 108). The external sensor (104) is a wrist-worn sensor integrated into a smart watch or fitness tracker (e.g., a heart rate sensor). The implanted sensor (108) is integrated into the housing of the OSA stimulation system (101). For example, the implanted sensor may be an oxygen sensor comprising an optical sensor in an IR/red transparent epoxy header. In this case, the implanted controller (102) is capable of wireless communication with the external sensor (104) and with an external controller (105). The external controller (105) may be, e.g., a dedicated controller with a text-based or graphical user interface, or software executed on a user's smart phone, tablet, computer, or other multi-purpose electronic device. The implanted controller (102) and/or the external controller (105) may be configured to communicate with one or more local, remote, or cloud-based servers. For example, in this case the external controller (105) is capable of communicating with a remote server (106) via intermediary cloud-based infrastructure (107).

In some aspects, the external controller (105) may be configured to execute a user application (109) configured to communicate with a clinical application (110) via an intervening cloud infrastructure (107), allowing a remote clinician to interact with the external controller (105) or the implanted controller (102). This configuration may allow for a clinician to view a user's sleep quality metric, to view sensor data, and to view and/or modify one or more settings of the OSA stimulation system. For example, the clinical may be able to editor stimulation profile or individual parameters stored on the external controller (105), which may in turn be transmitted to the implanted controller (102) in order to modify the treatment regimen or stimulation parameters applied by the OSA stimulation system (101).

In this exemplary embodiment, the implanted OSA stimulation system comprises a housing that includes both an implantable pulse generator (“IPG”), at least one implanted sensor (108) and a controller (102) configured to handle signal processing and storage, operation of the OSA stimulation system, and wireless communication between the OSA stimulation system (101) and a user application (109) executed on the external controller (105). The OSA stimulation system (101) further includes one or more electrodes (103) to deliver stimulation to one or more nerves which innervate an upper airway muscle of the human subject. As described herein, the system (100) may be configured to adjust one or more stimulation parameters based upon the detection and classification of respiratory events (e.g., apnea or hypopnea events) experienced by the human subject. In some aspects, the implanted controller (102) may be configured to detect and classify such respiratory events using a trained classifier executed by the implanted controller (102). The trained classifier may be used to analyze sensor data collected from any number of implanted or external sensors, using any of the techniques described herein. In alternative aspects, the classification may be performed by an external controller (105) or by a local, remote, or cloud-based server (e.g., it may be advantageous to offload the computation required for a classification to an external device, rather than using the power and limited processing capabilities of an implanted controller (102).

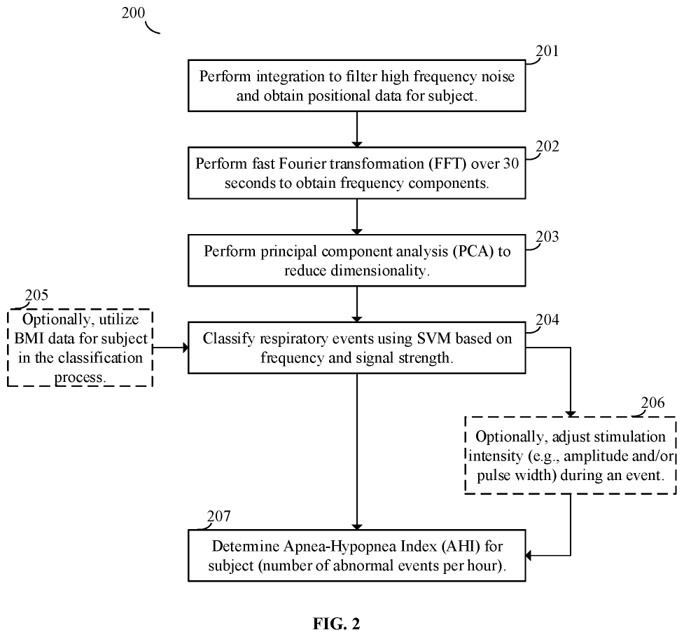

FIG. 2 is a conceptual flow diagram summarizing a method for detecting respiratory events (e.g., apnea or hypopnea events) using the systems described herein, and optionally the use of such systems to treat the subject for OSA. As illustrated by this figure, a respiratory activity signal (e.g., detected using an external or implanted acoustic sensor positioned on, within, or in proximity to the chest, bronchi, or trachea of the human subject) may be subjected to integration to filter high frequency noise and obtain positional data for subject (Step 201), and the resulting signal may in turn be subjected to a fast Fourier transformation (“FFT”) (e.g., over 20, 21, 22, 23, 24, 25, 26, 27, 28, 29, 30, 31, 32, 33, 34, 35, 36, 37, 38, 39, or 40 seconds, or within a range defined by any pair of the foregoing timepoints) to obtain frequency components (Step 203). The components may be subjected to a principal component analysis (“PCA”) to reduce dimensionality (Step 203) and then evaluated by a trained classifier (Step 204) (e.g., executed by the implanted controller (102), external controller (105) or by an application executed on any computer or other electronic device described herein.

As illustrated by this example, the trained classifier may be optionally configured to account for biomarker or physical data associated with the human subject; in this case body mass index (“BMI”) data is provided (Step 205). This resulting classification, produced using sensor data provided and any optional biomarker or physical data, may be stored to memory, transmitted to another device, output to a text-based or graphical user interface of any of the devices described herein, or used to adjust stimulation parameters. In this exemplary method, stimulation parameters for the OSA stimulation system are adjusted (Step 206) based on one or more respiratory events having been classified. For example, if the classifier detects a respiratory event and classifies the detected respiratory event as an apnea event, the system may be configured to titrate the amplitude and/or pulse width of stimulation to a higher level for the duration of the detected event. In some aspects, the system may be configured to titrate one or more stimulation parameters upwards or downwards in response to the detecting and classification of an event. The system may be configured to return to prior levels for one or more parameters, post-titration, e.g., when the classifier detects and classifies a normal breathing event.

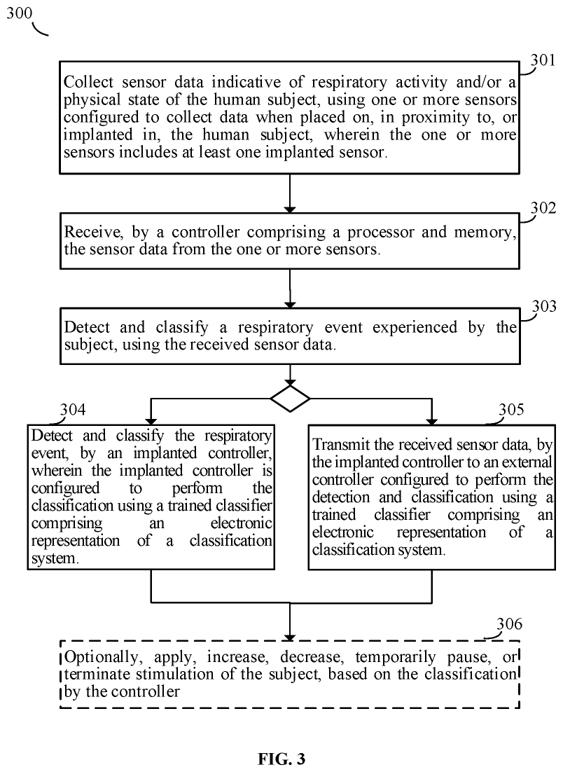

FIG. 3 is a conceptual flow diagram summarizing another general method for detecting and classifying respiratory events (e.g., as apnea or hypopnea events) using the systems described herein, and optionally adjusting OSA stimulation based on the same. As shown by this example, such methods may begin with the collection of sensor data indicative of respiratory activity and/or a physical state of the human subject, using one or more sensors configured to collect data when placed on, in proximity to, or implanted in, the human subject, wherein the one or more sensors includes at least one implanted sensor (Step 301). This sensor data may be provided to a controller comprising a processor and memory (Step 302). This controller may be an implanted or external controller, e.g., executed by an implanted controller (102) comprising a module of the implanted OSA stimulation system (101), software executed by an external controller (105), or executed by software on any other electronic device described herein (e.g., a local, remote, or cloud-based server). The controller may be configured to detect and classify a respiratory event using the received sensor data (Step 303). As illustrated by this flow diagram, the detection and classification process may be performed by an initial controller (e.g., an implanted controller (102)) that receives the sensor data from one or more sensors, as shown by Step 304. Alternatively, the sensor data may be transmitted from an initial controller to a second controller (or other electronic device, computer, server, etc.) for processing. In this case, an alternative processing workflow is depicted wherein the sensor data is transmitted to an external controller (105), e.g., a discrete controller of the OSA stimulation system configured to wireless communicate with an implanted controller (102) of the OSA stimulation system. As explained above, it may be preferable to offload the classification process to external hardware, rather than using the limited power and processing resources typically available to implanted electronic devices, such as an implanted controller (102). Finally, the resulting classification may be used to guide or select treatment parameters as shown by Step 306 (and explained in further detail in the analogous portion of FIG. 2).

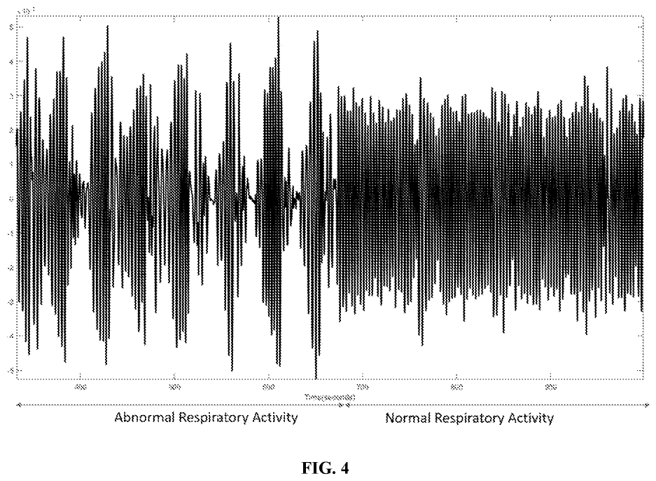

In some aspects, the systems and methods described herein may be configured to detect respiratory events by generating a respiratory waveform using data collected from one or more of the sensors described herein, and analyzing the respiratory waveform. During normal respiratory activity, the generated respiratory waveform will appear similar to a sinusoid with sustained amplitude. In contrast, during a respiratory event (abnormal respiratory activity), there is a suppression of chest activity followed by an oscillatory activity that corresponds to gasping of air to recover. The controller of the systems described herein may be configured to detect and/or classify respiratory events based upon this disruption in respiratory activity (e.g., a reduction in amplitude followed by oscillatory activity may be identified as a respiratory event). In some aspects, a controller may be configured to detect the amplitude of one or more peaks of the generated respiratory waveform (e.g., over a fixed or rolling window of time) in order to identify oscillatory activity. For example, if the peaks do not have a significant change in amplitude, then the signal may be classified as normal respiratory activity. In some aspects, a significant change may comprise a change of more than 20, 21, 22, 23, 24, 25, 26, 27, 28, 29, 30, 31, 32, 33, 34, 35, 36, 37, 38, 39, or 40%, or a change within a range bounded by any of the foregoing values, as compared to an average peak amplitude (e.g., for a window of time) or compared to a prior peak (e.g., the last peak detected, or a peak that occurred within 1, 2, 3, 4, or 5 seconds). If the amplitude of peaks decreases significantly (e.g., by 30%) and then continues to increases gradually (e.g., by 20, 21, 22, 23, 24, 25, 26, 27, 28, 29, 30, 31, 32, 33, 34, 35, 36, 37, 38, 39, or 40%, over the following 5, 6, 7, 8, 9, 10, 11, 12, 13, 14, or 15 peaks, that portion of the respiratory waveform may be classified as corresponding to abnormal respiratory activity. A respiratory waveform showing normal respiratory activity and abnormal respiratory activity in accordance with this aspect of the disclosure is provided as FIG. 4. This example illustrates a period of abnormal respiratory activity (left) followed by a period of normal respiratory activity (right). In many instances, such as the one shown in this example, abnormal activity may occur multiple times consecutively before normal breathing is restored. Accordingly, in some aspects the systems described herein may be configured to increase the amplitude of stimulation after the first detection of abnormal activity to help stimulate the subject out of a respiratory event.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators-such as “first,” “second,” “third,” etc. for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

What is claimed is:

1. A computer-implemented system for treating obstructive sleep apnea (“OSA”) in a human subject, comprising:
one or more sensors, wherein each sensor is configured to collect sensor data indicative of respiratory activity and/or a physical state of the human subject when placed on, in proximity to, or implanted in, the human subject, and wherein the one or more sensors includes at least one implanted sensor; and
a controller comprising a processor and memory, communicatively linked to the one or more sensors and configured to
receive the sensor data from the one or more sensors,
detect a respiratory event experienced by the subject, using the sensor data, and
classify the detected respiratory event, using a trained classifier comprising an electronic representation of a classification system; and
a stimulation system, communicatively linked to the controller and configured to deliver stimulation to a nerve which innervates an upper airway muscle of the human subject based on the classification by the controller.

2. The system of claim 1, where the controller is configured to classify the detected respiratory event as normal breathing, an apnea event, or a hypopnea event.

3. The system of claim 1, wherein the one or more sensors each comprise: a pressure sensor, an accelerometer, a sound sensor, a gyroscope, a heart rate monitor, an electrocardiogram (“ECG”) sensor, a blood pressure sensor, a blood oxygen level sensor, an electromyography (“EMG”) sensor, and/or a muscle sympathetic nerve activity (MSNA) sensor.

4. The system of claim 1, wherein the controller is further configured to generate a sleep quality metric for the human subject, wherein the sleep quality metric is based on the number of detected apnea or hypopnea events experienced by the subject.

5. The system of claim 4, wherein the sleep quality metric is an Apnea-Hypopnea Index (“AHI”), a Respiratory Disturbance Index (“RDI”), or a Respiratory Event Index (“REI”).

6. The system of claim 1, wherein the one or more sensors comprises a sub-clavically implanted inertial measurement unit (“IMTU”).

7. The system of claim 1, wherein the controller is located within a housing implanted in the human subject, and configured to
predict an airflow reduction amount and an oxygen desaturation level for the human subject using sensor data obtained from the one or more sensors.

8. The system of claim 1, wherein the one or more sensors comprises a sound sensor configured to detect a respiratory activity signal when positioned on, within, or in proximity to the chest, bronchi, or trachea of the human subject, and
wherein the controller is further configured to apply a filter to the respiratory activity signal, wherein the filter is configured to reduce or eliminate a component of the respiratory activity signal caused by the human subject's heartbeat and/or snoring activity.

9. The system of claim 7, wherein the filter comprises a Hilbert transform and wherein controller is configured to apply an adaptive threshold, using the trained classifier, to identify regions of the respiratory signal corresponding to apnea and/or hypopnea events.

10. The system of claim 7, wherein the one or more sensors comprises an IMU configured to detect motion by the human subject, and
wherein the controller is configured to identify one or more regions of the respiratory activity signal as a motion artifact based on detected motion by the human subject.

11. The system of claim 1, wherein the trained classifier was trained using a baseline dataset, wherein the baseline dataset comprises:
a) data generated during a prior single or multi-night polysomnography (PSG) study of the human subject; and/or
b) data generated from a prior single or multi-night PSG study of a population of human subjects.

12. The system of claim 4, wherein the system is configured to
a) output the sleep quality metric to a graphical or text-based interface of an electronic device; or
b) transmit the sleep quality metric to a local, remote, or cloud-based server.

13. The system of claim 1, wherein the controller is configured to detect the respiratory event experienced by the subject using sensor data received from at least or exactly 1, 2, 3, 4, 5, 6, 7, 8, 9, or 10 sensors.

14. The system of claim 1, wherein the controller is configured to cause the stimulation system to apply, increase, decrease, temporarily pause, or terminate the stimulation based on the classification by the controller.

15. The system of claim 13, wherein the controller is configured to cause the stimulation system to change an amplitude, pulse width, or frequency of the stimulation based on the classification by the controller.

16. A method for treating obstructive sleep apnea (“OSA”) in a human subject comprising:
collecting sensor data indicative of respiratory activity and/or a physical state of the human subject, using one or more sensors configured to collect data when placed on, in proximity to, or implanted within, the human subject, wherein the one or more sensors includes at least one implanted sensor;
receiving, by a controller comprising a processor and memory, the sensor data from the one or more sensors;
detecting a respiratory event experienced by the subject, using the received sensor data;
classifying the detected respiratory event, by the controller;
wherein the controller is configured to
perform the classification using a trained classifier comprising an electronic representation of a classification system, or
transmit the received sensor data to a server configured to perform the classification using a trained classifier comprising an electronic representation of a classification system; and
delivering stimulation to a nerve which innervates an upper airway muscle of the human subject based on the classification by the controller.

17. The method of claim 15, where the controller is configured to classify the detected respiratory event as normal breathing, an apnea event, or a hypopnea event.

18. The method of claim 15, wherein the one or more sensors each comprise: a pressure sensor, an accelerometer, a sound sensor, a gyroscope, a heart rate monitor, an electrocardiogram (“ECG”) sensor, a blood pressure sensor, a blood oxygen level sensor, an electromyography (“EMG”) sensor, and/or a muscle sympathetic nerve activity (MSNA) sensor.

19. The method of claim 15, wherein the controller is further configured to generate a sleep quality metric for the human subject, wherein the sleep quality metric is based on the number of detected apnea or hypopnea events experienced by the subject.

20. The method of claim 18, wherein the sleep quality metric is an Apnea-Hypopnea Index (“AHI”), a Respiratory Disturbance Index (“RDI”), or a Respiratory Event Index (“REI”).

21. The method of claim 15, wherein the one or more sensors comprises a sub-clavically implanted inertial measurement unit (“IU”).

22. The method of claim 15, wherein the controller is located within a housing implanted in the human subject, and configured to predict an airflow reduction amount and an oxygen desaturation level for the human subject using sensor data obtained from the one or more sensors.

23. The method of claim 15, wherein the one or more sensors comprises a sound sensor configured to detect a respiratory activity signal when positioned on, within, or in proximity to the chest, bronchi, or trachea of the human subject, and
wherein the controller is further configured to apply a filter to the respiratory activity signal, wherein the filter is configured to reduce or eliminate a component of the respiratory activity signal caused by the human subject's heartbeat and/or snoring activity.

24. The method of claim 15, wherein the controller is configured to cause the stimulation system to apply, increase, decrease, temporarily pause, or terminate the stimulation based on the classification by the controller.

25. The method of claim 23, wherein the controller is configured to cause the stimulation system to change an amplitude, pulse width, or frequency of the stimulation based on the classification by the controller.
